(19) United States
(12) Patent Application Publication
(10) Pub. No.: US 2024/0082564 A1
(43) Pub. Date: Mar. 14, 2024

(54) INTRA-AORTIC BALLOON PUMP ASSEMBLY

(71) Applicant: NuPulseCV, Inc., Raleigh, NC (US)

(72) Inventors: Joshua Ryan Woolley, Pittsburgh, PA (US); Robert Christopher Hall, Apex, NC (US); Duane Sidney Pinto, Newton, MA (US); Guruprasad Anapathur Giridharan, Louisville, KY (US)

(21) Appl. No.: 17/944,127

(22) Filed: Sep. 13, 2022

(51) Int. Cl. A61M 60/139; A61M 60/295; A61M 60/497; A61M 60/843

(52) U.S. Cl. CPC A61M 60/139; A61M 60/295; A61M 60/497; A61M 60/843; A61M 2205/32

(57) ABSTRACT

A blood pump assembly comprises a balloon defining an elongated inflatable chamber with an opening at the proximal end. The proximal region of the balloon is substantially cylindro-conically shaped, and tapering toward the proximal end of the balloon, the central region of the balloon is substantially cylindrically shaped with a substantially uniform exterior diameter. The distal region of the balloon is substantially cylindro-conically shaped, the distal region of the balloon tapering toward the distal end of the balloon. The length of the distal region of the balloon is greater than approximately 15% of the combined length of the proximal and central regions of the balloon. A driveline may be coupleable to the opening and have a connection element disposed at a proximal end of the driveline. A radio opaque marker may be integrated into the driveline at a distal end of the driveline.

### TECHNICAL FIELD

The present technology is directed to mechanical circulatory support devices and, in particular, to blood pump assemblies and associated devices, systems and methods.

### BACKGROUND

The prevalence of heart failure (HF) is increasing worldwide and is an expensive burden on health care providers. Despite advances in medical care, prognosis with HF remains poor, especially in advanced stages. Heart transplantation remains limited by the supply of donor organs. The use of left ventricular assist devices (LVAD) is stagnant at approximately 5,000 implants per year due to, among other things, the need for major operative intervention and the use of cardio-pulmonary bypass (CPB). Additionally, the high cost of these devices has prevented adoption in large potential markets, with some countries deciding not to fund the use of chronic LVADs.

Globally, the most common assist device for acute heart failure is the intra-aortic balloon pump (IABP), which is used clinically for limited time periods of several hours to several days. An IABP (also referred to herein as a “blood pump,” a “balloon” or an “expandable member”) is part of an IABP assembly which includes a driveline with two ends. One end is coupleable to the IABP and the other end is, often indirectly, coupleable to an external drive unit. The drive unit is the source of the working fluid (e.g., ambient air or helium), which is carried via the driveline to the IABP for inflation. The drive unit is also responsible for the deflation of the working fluid from the IABP, again via the driveline. Each year more than 150,000 patients worldwide receive IABP therapy. IABPs are much simpler than current LVADs, and the therapeutic effectiveness of counterpulsation therapy is well established. Counterpulsation requires no direct cannulation of the heart leading to easier implantation and explantation. Counterpulsation therapy is also less expensive compared to LVAD therapy.

Counterpulsation therapy is achieved by rapidly inflating the balloon immediately after aortic valve closure (dicrotic notch) and rapidly deflating the balloon just before the onset of systole. The dicrotic notch may be detected using a pressure sensor disposed at the tip of the IABP and the onset of systole may be detected using an electrocardiogram (ECG). Inflation and deflation, however, may both be triggered by either pressure sensor or ECG data. An example of an IABP with a pressure sensor disposed at the tip is the Arrow Ultra 8 Fiber-Optic IAB Catheter manufactured by Teleflex. The rapid inflation of the balloon increases the diastolic aortic pressure by 20-70%, improving end-organ and coronary perfusion. The rapid deflation of the balloon reduces the ejection pressure of the native ventricle, reducing afterload and left ventricular external work. Counterpulsation therapy has been shown to be most effective in patients when their systolic aortic pressures are between 40-70 mmHg, native heart rates between 80-110 bpm, and when the counterpulsation volume (i.e., balloon volume) equals the stroke volume of the native left ventricle.

IABPs have been used in HF patients awaiting transplant and in patients undergoing coronary artery bypass surgery. The balloon is generally implanted in the descending aorta with the driveline extending through the femoral artery. This implantation being sometimes referred to herein as the “femoral implantation” and the process resulting in the femoral implantation being sometimes referred to herein as the “femoral technique.” The femoral technique is a simple one as there are no significant arterial tortuosity or arterial curvature for the implanting clinician to deal with. However, IABPs implanted via the femoral technique require the patient to remain supine for the duration of therapy with the leg immobilized because (1) changes in orientation have been shown to diminish the effectiveness of therapy, (2) ambulation may cause the balloon or balloon driveline to kink due to movement of the leg leading to cyclic fatigue and ultimately failure of the balloon assembly, and (3) ambulation increases the risk of bleeding at the arterial access in the femoral artery. Consequently, patients cannot walk, or benefit from the IABP as an extended therapy for myocardial support. The lack of ambulation has been demonstrated to lead to poorer outcomes and prolong patient recovery and hospitalization. In addition to arterial access, biocompatibility, and durability issues limit the application of IABP to short durations (typically 2-4 days). While IABP support has been used for prolonged periods, the frequency of vascular complications, infections and bleeding are high.

More recently, IABPs have been implanted in the descending aorta with the driveline extending through the axillary/subclavian artery. This implantation being sometimes referred to herein as the “axillary implantation” and the process resulting in the axillary implantation being sometimes referred to herein as the “axillary technique.” When the IABP is implanted using the axillary technique, certain mobility issues related to femoral implantation are mitigated, facilitating patient ambulation. However, with axillary implantation, the natural buoyancy of the balloon when inflated can cause the balloon to flex and the balloon may undergo cyclic fatigue and fail. Axillary implantation has typically been performed surgically via entering the chest and cutting down to the axillary/subclavian artery.

Current IABPs are not optimally sized. IABPs are either too small for efficient therapy or are two long or wide, or otherwise misshaped, such that they obstruct the aorta itself or branching vessels. Further, current IABPs are not designed to navigate the tortuosity and curvature from the axillary artery to the thoracic aorta. In particular, conventional IABPs have drivelines that are stiff and do not have reinforcements. Similarly, the drivelines are not generally capable of withstanding the forces that are common when pulling a delivery dilator connected to an IABP driveline through the vasculature. This may lead to balloon malfunction, driveline kinks, and other increased adverse events when performing an axillary implementation of an IABP. The adverse events have been reported to be as high as 30% in clinical studies and include increased rates of balloon replacement and repositioning, bleeding, pseudoaneurysms, arterial dissection/damages, and hematomas. Finally, radio opaque material is typically added to an external surface of a driveline to provide a marker for properly disposing the balloon/driveline in the vasculature. But adding such material to an external surface of a driveline may create unwanted air pockets in the vasculature and may render it more difficult to pass the driveline through introducer sheathes for installation of the IABP (or conversely requires larger sized introducer sheathes than would otherwise be necessary).

### BRIEF DESCRIPTION OF THE DRAWINGS

Many aspects of the present technology may be better understood with reference to the following drawings. The components in the drawings are not necessarily to scale, and instead emphasis is placed on illustrating clearly the principles of the present disclosure.

FIG. 1 illustrates an embodiment of an IABP assembly implanted in the aortic arch vasculature in accordance with one embodiment of PiVAS technology.

FIGS. 2A-2D illustrate various stages of an exemplary procedure for intravascularly implanting an IABP assembly into a patient's vasculature that may be useful for the present disclosure.

FIGS. 3A-3C illustrate various exemplary features of an elongated delivery dilator that may be useful for the present disclosure.

FIGS. 4A-C illustrate various features of a driveline 116 that may be useful for the present disclosure.

FIG. 5 illustrates an exemplary planar depiction of a balloon pump assembly in accordance with an embodiment of the present disclosure.

FIG. 6 illustrates an exemplary close up of region 600 of the balloon pump assembly of FIG. 5.

FIG. 7A illustrates an exemplary planar illustration of the balloon of FIG. 5.

FIG. 7B illustrates an exemplary transverse cross section illustration of the balloon of FIG. 7A taken at section line 702 and facing a proximal region of balloon.

FIG. 8 illustrates an exemplary planar illustration of the driveline of FIG. 5.

FIG. 9 illustrates an exemplary transverse cross section of the driveline of FIG. 8 taken at section line 802 and facing a distal region of driveline.

FIG. 10 illustrates an exemplary longitudinal cross section of the driveline of FIG. 5 taken at a proximal end of the driveline.

FIG. 11 illustrates another exemplary longitudinal cross section of the driveline of FIG. 5 taken at a proximal end of the driveline.

FIG. 12 illustrates an exemplary longitudinal cross section of the driveline of FIG. 5 taken at a distal end of the driveline.

FIGS. 13A, 13B, and 14-15 illustrate planar illustrations of the balloon pump assembly of FIG. 5 having various exemplary support structures implanted within the balloon.

### DETAILED DESCRIPTION

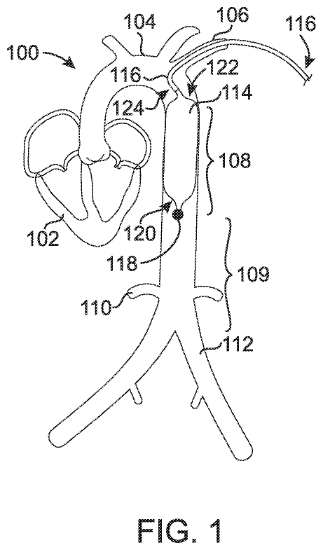

Specific details of several embodiments of the present technology are described herein with reference to FIGS. 1-15. Other applications and other embodiments in addition to those described herein are within the scope of the present technology. It should be noted that other embodiments in addition to those disclosed herein are within the scope of the present technology. Moreover, a person of ordinary skill in the art will understand that embodiments of the present technology may have configurations, components, and/or procedures in addition to those shown or described herein and that these and other embodiments may be without several of the configurations, components, and/or procedures shown or described herein without deviating from the present technology. Reference throughout this description to “one embodiment,” “an embodiment,” one or more embodiments,” an “nth embodiment,” “some embodiments,” or a phrase of similar effect means that a particular feature, structure or characteristic described in connection with the embodiment is included in at least one embodiment of the present disclosure. Thus, use of such terminology is not necessarily referring to the same embodiment. For example, it is expressly contemplated that the features described herein may be combined in any suitable manner in one or more embodiments.

Related Technologies

Applicant/Assignee NuPulseCV, Inc. of Raleigh, North Carolina has developed a percutaneously-delivered intravascular ventricular assist system (PiVAS) that functions as a chronic counterpulsation device as generally described in U.S. patent application Ser. No. 16/876,110, the contents of which are incorporated herein by reference in its entirety. The PiVAS includes an expandable member that is implanted in the descending aorta via an improved axillary technique using minimally invasive surgical techniques. When implanted, the driveline of the PiVAS extends through the axillary or subclavian artery. The PiVAS offers an alternative therapy for HF patients by providing partial circulatory support that may be implanted minimally invasively without entering the chest and does not require cardiopulmonary bypass (CPB) or blood products.

NuPulseCV has also developed: (1) an IABP assembly with one or more sensors (the “Sensor Technology”), generally described in a U.S. Patent Application filed on the same date as the present disclosure, entitled “Intra-Aortic Balloon Pump Assembly with Pressure Sensor,” identifying Joshua Ryan Woolley, Sonna Manubhai Patel-Raman, Guruprasad Anapathur Giridharan as inventors, and having attorney-docket number 8018US00/236533-30034; and (2) a blood pump support structure for a blood pump assembly (the “Blood Pump Support Structure Technology”), generally described in a U.S. Patent Application filed on the same date as the present disclosure, entitled “A Blood Pump Support Apparatus and Method for a Blood Pump Assembly,” identifying Robert Christopher Hall, Joshua Ryan Woolley, Guruprasad Anapathur Giridharan, and Duane Sidney Pinto as inventors, and having attorney-docket number 8020US00/236533-30035, the contents of both applications are incorporated by reference herein in their entirety. The Sensor Technology solves certain problems associated with, among other things, axillary implantation of a IABP assembly using a pressure sensor disposed at the tip (or distal end) of the IABP, and the Blood Pump Support Structure Technology solves certain problems associated with, among other things, the blood pump rolling or folding upon itself or otherwise developing crevices when in operation (e.g., when disposed in a descending aorta).

FIG. 1 illustrates an embodiment of an IABP assembly implanted in the aortic arch vasculature 100 in accordance with one embodiment of the PiVAS technology. The aortic arch vasculature 100 includes heart 102, the aortic arch 104, the axillary/subclavian artery 106, the thoracic aorta 108, the abdominal aorta 109 (collectively with the thoracic aorta 108, the descending aorta 108-109), the two branches of the renal artery 110, and the two branches of the common iliac artery 112. The thoracic aorta 108 starts at approximately the axillary/subclavian artery 106 and extends to approximately the abdomen. The abdominal aorta 109 starts at approximately the abdomen and extends to approximately the two branches of the common iliac artery 112. The IABP assembly may include IABP or balloon 114 and driveline 116, where the balloon 114 is disposed in the thoracic aorta 108. The balloon 114 may include marker 118 disposed at the distal end 120 of balloon 114, the marker 118 being made of a radio-opaque material suitable to track the position of balloon 114 during radiological intervention (e.g., on an x-ray or fluoroscopy).

In an embodiment, balloon 114 may be a pneumatically driven expandable member defining an elongated inflatable chamber, the balloon 114 being designed for long-term biocompatibility and safety. The proximal end 122 of the balloon 114 may be coupled to a distal end 124 of the driveline 116. In one embodiment, the proximal end 122 of the balloon 115 may include an engagement region that is sized and shaped to fit over a distal end 124 of the driveline 116. An attachment feature such as a compression ring or other suitable element providing an airtight connection/pneumatic seal between the engagement region and driveline may be used to couple the balloon 114 to the driveline 116. The proximal end of the driveline 116 may be coupled to a drive unit (e.g., an external driver), not depicted.

The balloon 114 may be composed of a biocompatible, non-thrombogenic elastomeric material (e.g., Biospan®-S) or other suitable materials and may have features described in U.S. Pat. No. 8,066,628, the disclosure of which is incorporated hereby by reference in its entirety. In an embodiment, the maximum device displacement volume may be closely matched to the stroke volume of the heart and is a parameter that may be varied to provide effective counterpulsation therapy. The balloon 114 may have a displacement volume of between about 20 ml and about 60 ml. In some embodiments, the displacement volume is about 50 ml. The balloon 114 may have a length between about 15 cm and about 30 cm.

The balloon may be disposed in the descending aorta 108-109 (e.g., the thoracic aorta 108) via an improved axillary technique wherein, when implanted, the driveline 116 extends through the axillary/subclavian artery 106. As described, below, an arterial skin interface/stopper device may be utilized to enable long-term implant using minimally invasive surgical techniques. The balloon 114 may be non-obstructive and may lay at least substantially flat in the thoracic aorta 108 without folds or crevices when deflated. This enables the device (e.g., the IABP assembly or the PiVAS system) to be turned off for prolonged periods, e.g., patients may routinely stop the device for 60-plus minutes with no adverse consequences and the balloon 114 may have a durability for over 2.5 years of use.

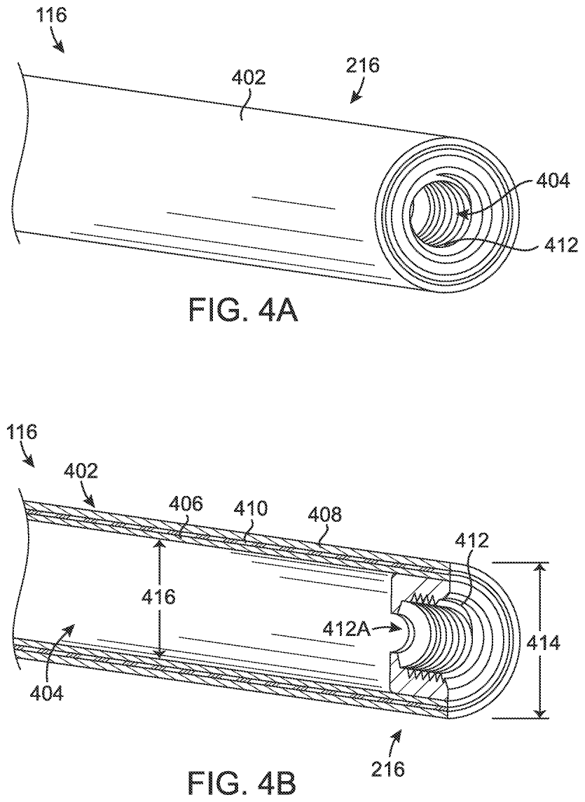

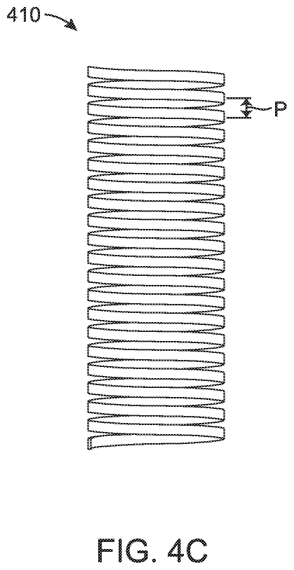

The driveline 116 may be a thin (e.g., 4.2 mm outer diameter) driveline that shuttles a working fluid (e.g., air, gas, etc.) between the balloon 114 and the drive unit (not depicted) via a lumen (FIG. 4B, element 404). The relatively small diameter of the driveline 116 compared to the relatively large axillary/subclavian artery 106 mitigates risk of limb ischemia. The driveline 116 may include an inner driveline and an outer driveline. The inner driveline may be an elongated support structure having a lumen extending there through for delivering the working fluid to and from the balloon 114. The inner driveline may be positioned at least partially within the patient's vasculature (e.g., between the aorta and the axillary/subclavian artery). After implantation, the inner driveline may have a distal end portion 124 coupled to the balloon 114 and positioned within the patient's vasculature (e.g., the thoracic aorta 108) and a proximal end portion coupled to the outer driveline and positioned external to the patient's vasculature at an arteriotomy in, for example, an axillary artery/subclavian artery 106, or other suitable blood vessel. The proximal end portion of the inner driveline may be coupled to a distal end of the outer driveline using any suitable technique (e.g., compression rings, suturing, gluing, stitching, etc.).

An arterial interface device or stopper device (“AID”) may be used to provide hemostasis at an arteriotomy where the inner driveline exists the vasculature. The AID may include one or more anchoring elements used to secure the device in a desired orientation or position, and one or more ports. For example, one port might provide wire axis to the patient's vasculature. The AID may have features similar to those described in U.S. Pat. No. 7,892,162, the disclosure of which is incorporated herein by reference in its entirety. The AID may be deployed or advanced over the inner driveline (e.g., that portion that is externalized from the vasculature) through a shaft in the AID that defines a lumen. The inner driveline may be inserted into and extend through the shaft. The outer driveline may be coupled to the inner driveline at a position proximate the AID.

The outer driveline may also be an elongated structure having a lumen extending there through. The outer driveline may be positioned at least partially subcutaneously but external to the patient's vasculature. After implantation, the proximal end portion of the outer driveline may be coupled to a skin interface device (described below). The lumen of the driveline (e.g., inner and outer drivelines) may be coupled to the lumen of the blood pump to transport the working fluid to and from the blood pump.

The drive unit may be a small, portable device that actuates the balloon pump by generating a flow of working fluid (e.g., a gas or other fluid such as ambient air or helium) into and out of the balloon 114 via the driveline 116. For example, the drive unit may generate a positive pressure to accelerate the working fluid into the balloon 114, thereby inflating the balloon 114 and may induce a negative pressure to withdraw the working fluid from the balloon 114, thereby deflating balloon 114. The drive unit may utilize a bellows, a blower, a compressor, an accelerator, or other similar features to direct the flow of working fluid into and out of the balloon 114. The drive unit may have a feature to prevent over-inflation of the balloon 114.

A skin interface device (“SID”) may be a transcutaneous device that enables the drive unit to drive operation of the balloon. The SID may provide a stable and/or secure exit site for the driveline 116 (e.g., the outer driveline). In an embodiment where the driveline 116 comprises an inner driveline and outer driveline, the proximal end of the outer driveline may be coupled to an internal facing portion of SID and the drive unit may be coupled to an external facing portion of the SID. As such, SID may direct gases received from the drive unit to the outer driveline for delivery to the balloon 114. SID may be similar to the interface devices described in U.S. Pat. No. 10,137,230, the disclosure of which is incorporated herein by reference in its entirety.

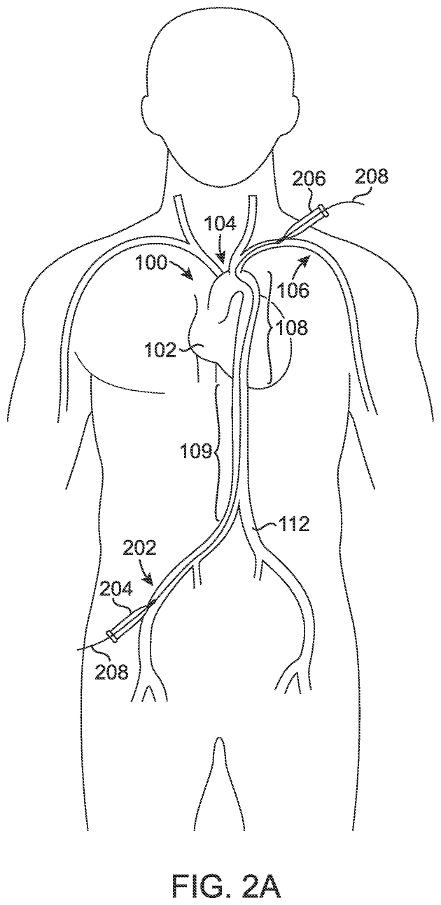

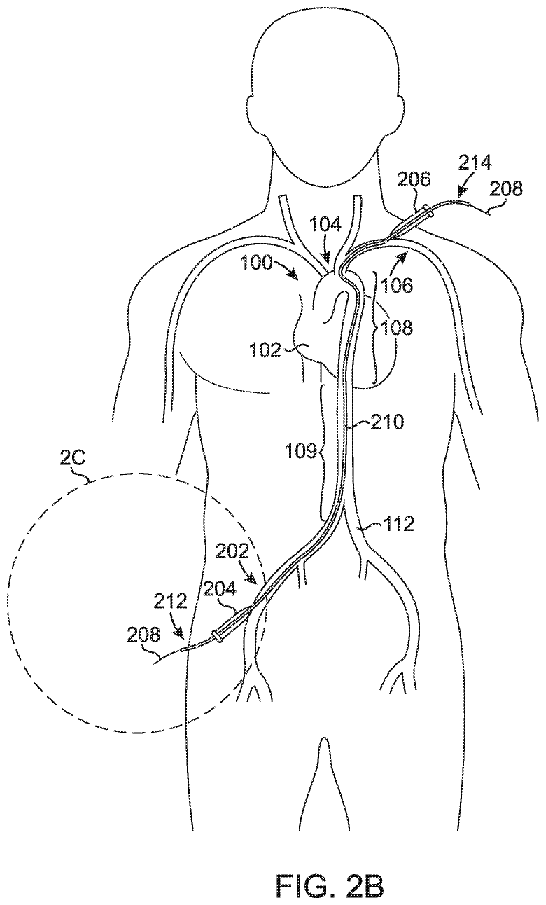

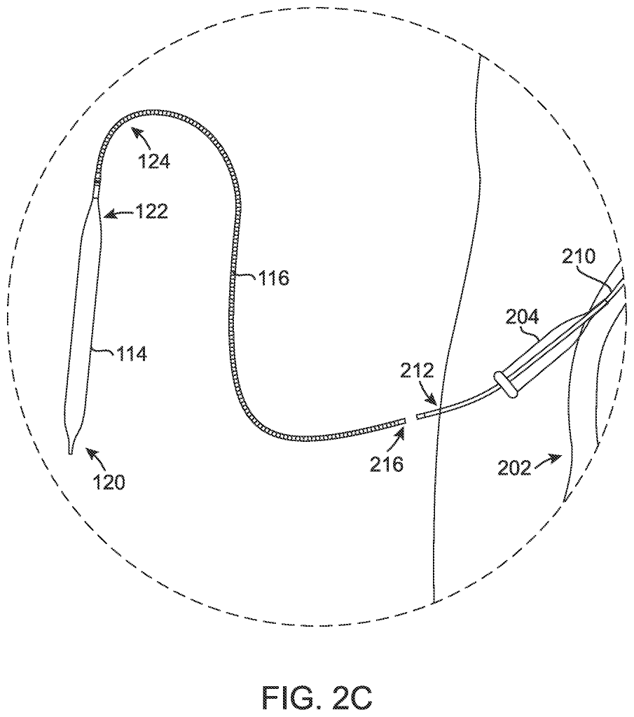

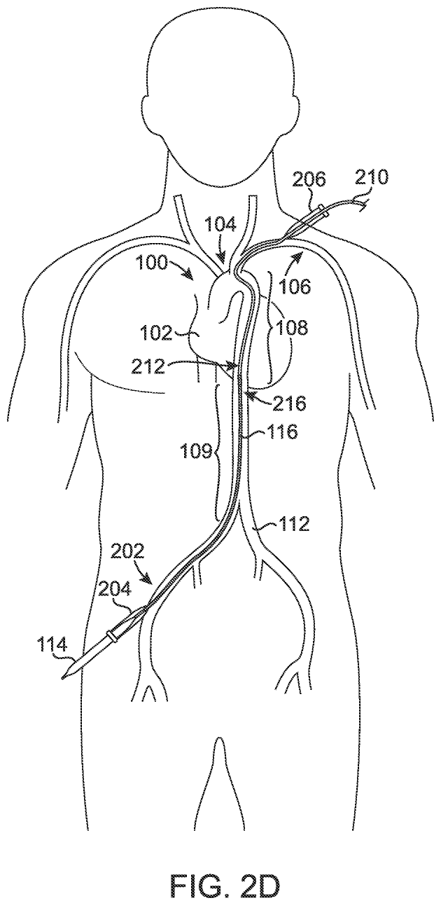

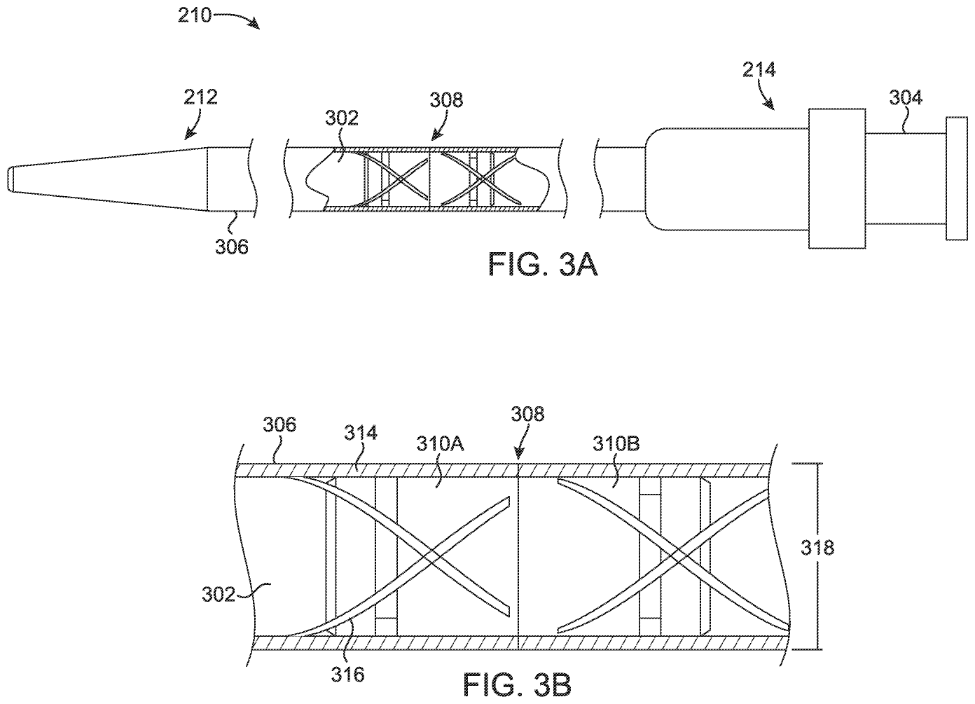

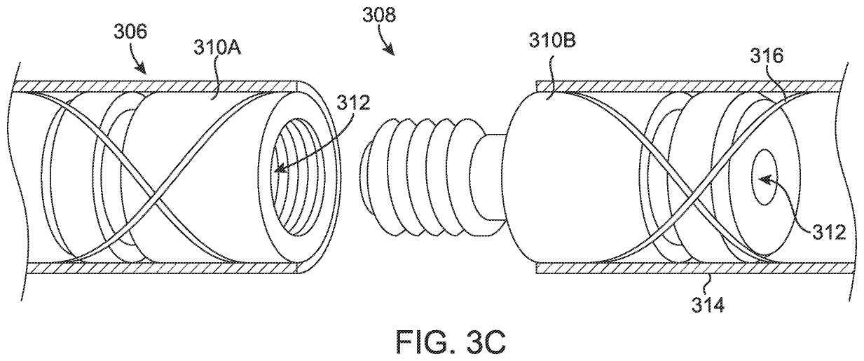

The balloon 114 may be implanted via the femoral artery and explanted via the axillary/subclavian artery 106 without the need to enter the chest. In one embodiment, the intravascular implant procedure is as described in U.S. patent application Ser. No. 16/876,110. Briefly and with reference to FIGS. 2A-2D, which illustrate various stages of a procedure for intravascularly implanting an IABP assembly into a patient's vasculature, introducer sheaths 204, 206 may be inserted into the femoral artery 202 and the axillary artery and/or subclavian artery 106, respectively. A guidewire 208 may be introduced into either introducer sheath 204, 206 and advanced through the patient's vasculature such that the guidewire 208 extends between the femoral artery 202 and the axillary artery/subclavian artery 106, thereby establishing a “rail.” This process may be referred to as “flossing” the patient. After the rail is established, an elongated delivery dilator (sometimes referred to as a delivery shaft, a delivery catheter, or an interceptor dilator) 210 may be advanced over the guidewire 208 to extend between the introducer sheathes 204, 206. In one embodiment, the elongated delivery dilator has a small lumen that extends throughout that is sized to allow the guidewire 208 (e.g., a 0.035 inch guidewire) to slide there through. In one embodiment and with reference to FIG. 3, the elongated delivery dilator 210 may have a inwardly slowed or pointed distal end region to help guide it into the introducer sheath 204, 206 (e.g., introducer sheath 206 disposed at the axillary or subclavian artery 106) and the delivery dilator 210 is advanced through the vasculature such that a proximal end 214 of the delivery dilator 210 is externally accessible at the axillary or subclavian artery 106 and a distal end 212 of the delivery dilator 210 is externally accessible at the femoral artery 202. The guidewire 208 may be removed once the delivery dilator 210 is so positioned.

The externalized distal end 212 portion of the elongated delivery dilator 210 may then be releasably connected to the driveline 116 using a connector or other mechanism. For example, the distal end 212 of the delivery dilator 210 may have a threaded male connection element and the proximal end 216 of the driveline 116 may have a threaded female connection element sized and shaped to interface with the threaded male connection element of the distal end 212 of the delivery dilator 210. The distal end 212 of the delivery dilator 210 may then secured to the proximal end 216 of the driveline 116 by screwing the threaded male connection element into the threaded female male connection element, as is depicted at FIG. 2C. For the avoidance of doubt, FIG. 2C depicts the threaded connection elements just prior to connection and after guidewire 208 is removed.

In one embodiment, the distal end 212 of the delivery dilator 210 includes a removable portion that covers the threaded male connection element, the removable portion optionally having the inwardly slowed or pointed end region noted above. In such an embodiment, a practitioner may remove the removable portion after the distal end 212 of the delivery dilator 210 is externalized via the introducer sheath 204 at the femoral artery 202 to expose the threaded male connection element.

The driveline 116 and balloon 114 may then be moved into the patient's vasculature by pulling on the proximal end 214 of the delivery dilator 210 that is externalized at the axillary and/or subclavian artery 106. In the process, the driveline 116 is first pulled into the body through the introducer sheath 204 at the femoral artery 202. The driveline 116 may be externalized through the introducer sheath 206 at the axillary artery and/or subclavian artery 106 and the delivery dilator 210 may be unscrewed. The balloon 114 may be pulled into the introducer sheath 204 at the femoral artery 202 as the driveline 116 is pulled out of the axillary and/or subclavian artery 106, moving the balloon 114 through the vasculature in a direction opposite to blood flow. The practitioner continues to pull the driveline 116 until the balloon 114 is in a desired position (e.g., within the thoracic aorta 108). The positioning of the balloon 114 may be confirmed via bony landmarks form CT and/or via contrast angiography using, for example, a 5F Omni™ Flush or pigtail catheter inserted via the introducer sheath 204 at the femoral artery 202. The foregoing was described in reference to an embodiment where the driveline 116 is at least as long as the vasculature from the femoral artery 106 to the axillary artery and/or subclavian artery 106. In an embodiment where the driveline 116 is not sufficiently long to extend such vasculature, the balloon 114 may be pulled into the introducer sheath 204 at the femoral artery 202 before the driveline 116 is pulled out of the axillary and/or subclavian artery 106, and the delivery dilator 210 may not be unscrewed until later in the pulling process (e.g., until the balloon 114 is in the desired position, for example, in the thoracic aorta 108).

In some embodiments, balloon 114 may be de-aired before pulling it into the vasculature (e.g., femoral artery 202) via an introducer sheath 204, 206 (e.g., introducer sheath 204 at femoral artery 202). For example, a practitioner may attach a Y-connector to the proximal end 214 of the elongated delivery dilator 210. Because the lumen of the elongated delivery dilator 210 is in fluid communication with an interior of the balloon 114 (via the driveline 116), the practitioner may use a syringe or other pump element to remove any air from the balloon 114. The practitioner may then close off the Y-connector (e.g., using a stopcock) to maintain a negative pressure in the balloon 114, the driveline 116, and the elongated delivery dilator 210. Without being bound by theory, de-airing the balloon 114 is expected to reduce and/or minimize the profile of the balloon 114, making it easier to pull through the introducer sheath 204, 206 and into the patient's vasculature.

In some embodiments, the balloon 114 is folded, twisted, or otherwise placed in a delivery state with a decreased cross-sectional area before or while being pulled into the femoral artery 202 via the introducer sheath 204. For example, the balloon 114 may be pulled through a delivery sheath such as a funnel assembly or other folding tube to decrease one or more dimensions of the balloon 114. Exemplary delivery sheaths are described in U.S. patent application Ser. No. 16/876,110. In some embodiments, the balloon 114 may be manually folded or twisted in addition to, or in lieu of, using a delivery sheath. Reducing a dimension (e.g., a cross-sectional dimension, an outer diameter, etc.) of the balloon 114 may facilitate pulling the balloon 114 through an introducer sheath 204, 206 and into the patient's vasculature. In some embodiments, the delivery sheath may be docked on to an introducer sheath 204, 206 (e.g., the introducer sheath 204 at the femoral artery 202). The delivery sheath may (1) keep balloon 114 folded and compact during insertion; (2) be configured to permit balloon 114 defeat a hemostatic valve on the introducer sheath 204, 206 with which it is docked, and (3) be configured such that an inner diameter of the delivery sheath is substantially identical to an inner diameter of the introducer sheath 204, 206 with which it is docked so that during insertion the balloon 114 experiences a smooth or seamless transition from delivery sheath to introducer sheath 204, 206 (i.e., from the perspective of the balloon 114, during insertion, it is passing through the same conduit/lumen). In some embodiments, the folding and/or twisting may be done in combination with the de-airing procedure described above. In other embodiments, the folding and/or twisting may be done in lieu of the de-airing procedure described above. In some embodiments, the folding procedure may be performed immediately before pulling the balloon 114 into the vasculature. In other embodiments, the balloon 114 may come pre-loaded in the delivery sheath. In some embodiments, however, no delivery sheath is used and the balloon 114 is directly loaded/pulled into and through an introducer sheath 204, 206.

With reference to FIG. 3A-3C, various features of the elongated delivery dilator 210 are depicted. The delivery dilator 210 forms a generally tubular conduit, shaft, pipe, or the like having a lumen 302 extending there through. The lumen 302 is sized such that the elongated delivery dilator 210 may be advanced over guidewire 208 (e.g., a 0.035 inch wire). As previously described, the elongated delivery dilator 210 may include a proximal end 214 and a distal end 212. In some embodiments, the proximal end 214 may include a valve or port 304 (e.g., a check valve, a Luer lock, a sealable silicone port, etc.) for providing controlled access to the lumen 302. For example, a syringe, Y-connector, 3-way stopcock, or the like may be attached to the driveline 116 at the valve 304 to de-air the balloon 114 before pulling it into the patient's vasculature, as previously described. In embodiments including the port 304, the port 304 remains external to the patient (e.g., at the axillary artery access site) throughout the implant procedure. The distal end 212 of the elongated delivery dilator 210 may be designed to penetrate a hemostatic valve in an introducer sheath (e.g., introducer sheath 206) when inserted into the introducer sheath in a retrograde direction. For example, in the illustrated embodiment the distal end 212 is at least partially pointed and/or inwardly sloped to help it penetrate a hemostatic valve.

The distal end 212 of the elongated delivery dilator 210 may include a removable portion 306 (e.g., a removable distal tip). The removable portion 306 may be secured to the elongated delivery dilator 210 at an attachment interface 308, described in detail below with reference to FIGS. 3B and 3C. During the intravascular implant procedures described herein, the removable portion 306 is removed from the elongated delivery dilator 210 after the distal end 212 is externalized at the introducer sheath 204 proximate to the femoral artery 202. The driveline 116 is then connected to the elongated delivery dilator 210 at the attachment interface 308.

FIGS. 3B and 3C illustrate additional features of the elongated delivery dilator 210 proximate the attachment interface 308. More specifically, FIG. 3B illustrates the removable portion 306 connected to the elongated delivery dilator 210 at the attachment interface 308, and FIG. 3C illustrates the removable portion 306 spaced apart (e.g., disconnected) from the elongated delivery dilator 210 at the attachment interface 308. Referring to FIGS. 3B and 3C together, the elongated delivery dilator 210 includes a first connection element 310A and a second connection element 310B proximate the attachment interface 308. The first connection element 310A and the second connection element 310B engage to releasably secure the removable portion 306 to the elongated delivery dilator 210. In the illustrated embodiment, the first connection element 310A is a threaded female connection element and the second connection element 310B is a threaded male connection element. In operation, the removable portion 306 may be releasably secured to the elongated delivery dilator 210 by screwing the threaded male connection element into the threaded female connection element. The removable portion 306 may be removed from the elongated delivery dilator 210 by unscrewing the threaded female connection element from the threaded male connection element. In other embodiments, the first connection element 310A is a threaded male connection element and the second connection element 310B is a threaded female connection element. Regardless, the first connection element 310A and the second connection element 310B may include apertures 312 extending there through to allow the elongated delivery dilator 210 to be advanced over a wire.

The first connection element 310A and the second connection element 310B may be composed of a generally inflexible material (e.g., stainless steel). Because the first connection element 310A and the second connection element 310B are composed of a generally inflexible material, the elongated delivery dilator 210 may not bend or flex at the attachment interface 308. Therefore, to ensure that the elongated delivery dilator 210 may be routed through various curves in the patient's vasculature despite the stiffness at the attachment interface 308, the first connection element 310A and the second connection element 310B may have a relatively short combined length when connected. For example, the first connection element 310A and the second connection element 310B may have a combined length less than about 2 cm and/or less than about 1 cm.

FIGS. 4A-C which illustrate various features of a driveline 116. FIGS. 4A and 4B are perspective and cross-sectional views, respectively, of the driveline 116. As shown in FIGS. 4A and 4B, the driveline 116 may have a generally tubular wall 402 defining a lumen 404 extending there through. The generally tubular wall 402 may be gas impermeable to prevent gases from leaking out of the lumen 404 and into the patient's vasculature. In some embodiments, the generally tubular wall 402 may comprise a plurality of layers. In the illustrated embodiment, for example, the generally tubular wall 402 includes an inner membrane 406 and an outer membrane 408 concentrically enclosing the inner membrane 406. A coil or other helically wound kink-resistant element 410 (shown alone in FIG. 4C) may be disposed between and/or partially within the inner membrane 406 and the outer membrane 408. The inner membrane 406 and the outer membrane 408 may be composed of an at least partially flexible material. Furthermore, the outer membrane 408 may be composed of an antithrombogenic material, such as elastin-s, to reduce and/or prevent clot formation on the driveline 116 when it is implanted in a patient's vasculature. The coil 410 may be composed of Nitinol or another suitable kink-resistant material such as stainless steel. In one embodiment coil 410 is a biocompatible material. In some embodiments, the coil 410 may have a pitch P less than about 1 mm (e.g., about 0.5 mm). Without being bound by theory, incorporation of the coil 410 into the tubular wall 402 provides several advantages, including, (1) providing added stability to the generally tubular wall 402, (2) reducing the required thickness of the generally tubular wall 402 to achieve a desired stability, and (3) reducing and/or preventing the driveline 116 from kinking or collapsing. In some embodiments, the coil 410 only extends along a portion of the driveline 116, dividing the driveline 116 into a “reinforced” section and an “unreinforced” section. In other embodiments, the coil 410 extends along the entire or approximately the entire length of the driveline 116.

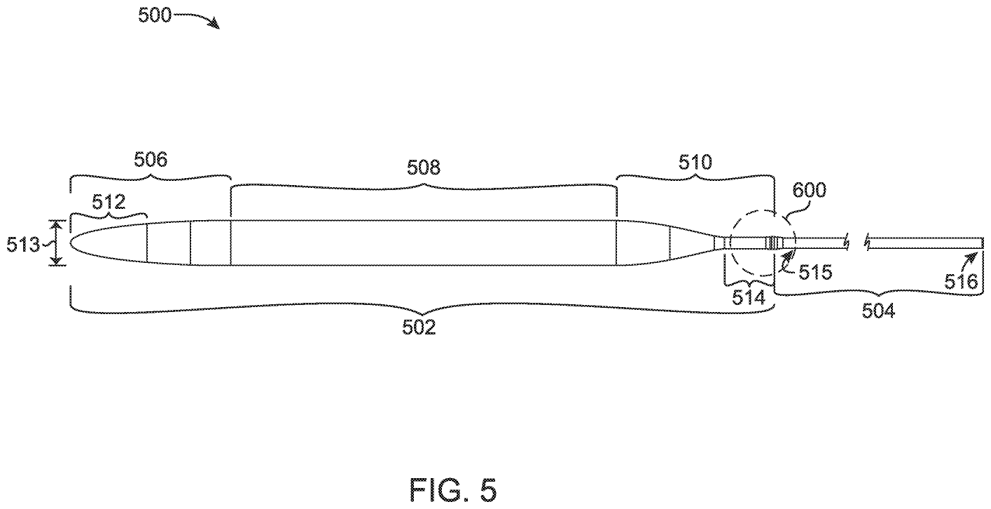

The proximal end 216 of the driveline 116 may also include a connection element 412 for connecting the driveline 116 to the elongated delivery dilator 210 (FIGS. 5A-5C). Accordingly, the connection element 412 may also be referred to as the elongated delivery dilator connection element 412. The connection element 412 may be a threaded female connection element that is sized and shaped to receive corresponding threaded male connection element 310B on the elongated delivery dilator 210. In other embodiments, the connection element 412 may be a threaded male connection element that is sized and shaped to receive a corresponding threaded female connection element on the elongated delivery dilator 210. In yet other embodiments, the connection element 412 is another suitable fastening mechanism to secure the first driveline 120 to the elongated delivery dilator 280. In some embodiments, the connection element 412 includes an aperture 412A or other feature that permits air to flow through and/or around the connection element 412 and into the lumen 404. Thus, when the driveline 116 is connected to the elongated delivery dilator 210 via the connection element 412, gas may flow through the elongated delivery dilator 210 and into the lumen 404 via the aperture 412A, and vice versa.

The driveline 116 may have an outer diameter 414 that is substantially uniform. In one embodiment the outer diameter 414 of driveline 116 is less than about 6 mm. For example, in some embodiments the outer diameter 414 is about 4.2 mm. In other embodiments, the outer diameter 414 is less than about 4.2 mm. In particular, the outer diameter 414 of the driveline 116 may be selected such that it fits through one or both of the introducer sheaths 204, 206 during the intravascular implant procedures described herein. For example, in some embodiments the driveline 116 has an outer diameter of 4.2 mm (e.g., 12.6 Fr), the introducer sheath 204 proximate the femoral artery 202 is a 16 Fr sheath, and the introducer sheath 206 proximate the axillary and/or subclavian artery 106 is a 14 Fr sheath. In such embodiments, the driveline 116 will fit through both introducer sheaths 204, 206. The outer diameter 214 may also be small enough to fit within varying patient anatomy (e.g., larger drivelines may only be implanted in patients with axillary artery diameters above a certain threshold). The driveline 116 may have an inner diameter 416 (i.e., the diameter of the lumen 404, defined by the inner membrane 406) between about 2 mm and 5 mm. For example, in some embodiments the inner diameter 416 is about 3.3 mm. In other embodiments the inner diameter 416 is less than 3.3 mm.

The driveline 116 may have a relatively long length. For example, the driveline 116 may be greater than about 100 cm (e.g., about 150 cm). For example, having a relatively long length ensures that a portion of the driveline 116 remains external to the patient during the implant procedure. If the driveline 116 becomes disconnected from the elongated delivery dilator 210 during the implant procedure, the portion of the driveline 116 that remains external to the patient may be used to retrieve the driveline 116 and the balloon 114 from the patient's vasculature. Because of the relatively long length, the driveline 116 is generally cut before securing the driveline 116 to a second driveline (not depicted). For example, a portion of the proximal end 222 may be removed, thereby removing the portion of the driveline 116 having the connection element 412, which is no longer needed because the driveline 116 is in position before cutting the driveline 116.

In embodiments in which only a portion of the driveline 116 is reinforced, the reinforced portion may have a first outer diameter that is greater than a second outer diameter of the unreinforced portion. For example, the reinforced portion may have an outer diameter of about 4.2 mm and the unreinforced portion may have an outer diameter of about 4 mm. In such embodiments, the reinforced portion may have a length greater than about 30 cm and the unreinforced portion may have a length greater than about 10 cm. In some embodiments, the transition between the first and second outer diameters may be tapered or otherwise gradual.

Returning to FIGS. 3A-C, the second connection element 310B may be used to connect the driveline 116 to the elongated delivery dilator 210 (once the removable portion 306 is removed) by engaging the elongated delivery dilator connection element 412 on the driveline 116. Accordingly, the second connection element 310B may also be referred to as the driveline connection element 312B. In embodiments in which the elongated delivery dilator connection element 412 on the driveline 116 is a threaded male connection element, the second connection element 310B is a threaded female connection element. Likewise, in embodiments in which the elongated delivery dilator connection element 412 is a threaded female connection element, the second connection element 310B is a threaded male connection element. The aperture 312 on the second connection element 310B aligns with the aperture 412A on the elongated delivery dilator connection element 412 to place the elongated delivery dilator 210 in fluid connection with the lumen 404 of the driveline 116 (and an internal volume of the balloon 114. This permits gases to be withdrawn from the balloon 114 and through the driveline 116 and the elongated delivery dilator 210 during, for example, a de-airing procedure.

The elongated delivery dilator 210 may include an outer membrane 314. The outer membrane 314 may be gas impermeable and may be composed of any suitable biocompatible and/or anti-thrombogenic material. For example, in some embodiments the outer membrane 314 is composed of elastin-S. In some embodiments, the elongated delivery dilator 210 also includes one or more reinforcement wires 316. In some embodiments, the reinforcement wires 316 are generally similar to the coil 410 in the driveline 116. For example, the reinforcement wires 316 may be one or more helically wound Nitinol coils that are generally kink-resistant. Although only shown proximate the attachment interface 308, the reinforcement wires 316 may extend along a full length or a substantial length of the elongated delivery dilator 210. A length is a substantial length of the elongated delivery dilator if it is within 95%, 90%, 85%, 80%, 75%, 70%, 65%, 60%, 55%, 50%, 45%, 40%, 35%, 30%, 25%, 20%, 15%, or 10% of the elongated delivery dilator 210. In other embodiments, however, the reinforcement wires 316 are only located proximate the attachment interface 308 to provide added stability at the attachment interface 308.

The elongated delivery dilator 210 may have an outer diameter 318 less than about 6 mm (excluding the port 304 in embodiments in which it is included). For example, in some embodiments the outer diameter 318 is about 4.2 mm. In other embodiments, the outer diameter 318 is less than about 4.2 mm. In particular, the outer diameter 318 of the elongated delivery dilator 210 may be selected such that it fits through the one or both introducer sheaths 204, 206 during the implant procedures described herein. For example, in some embodiments the elongated delivery dilator 210 has an outer diameter of 4.2 mm (e.g., 12.6 Fr), the introducer sheath 204 proximate the femoral artery 202 is a 16 Fr sheath, and the introducer sheath 206 proximate the ancillary and/or subclavian artery 106 is a 14 Fr sheath. Accordingly, elongated delivery dilator 210 may fit through both the introducer sheaths 204, 206. The outer diameter 318 may also be small enough to permit the elongated delivery dilator 210 to fit within and through varying patient anatomy. In some embodiments, the elongated delivery dilator 210 has an outer diameter 318 that is the same and/or about the same as the outer diameter 414 of the driveline 116. In one embodiment, the elongated delivery dilator 210 also has a relatively long length. For example, the elongated delivery dilator 210 may have a length that is greater than about 100 cm (e.g., about 150 cm). As described above, having a relatively long length ensures that the elongated delivery dilator 210 may extend between the introducer sheath 204 and the introducer sheath 206.

The balloon 114 may be triggered, at least in part, by pressure sensor data (e.g., from pressure sensor element associated with balloon 114 and/or the patient's ECG via surface ECG sensors. The associated ECG sensors may be coupled to the SID via ECG leads, which relay the measurements received from the sensors to the drive unit via a wired or wireless connection. In other embodiments, the ECG sensors may be wirelessly connected to the drive unit and may transmit the sensed measurements directly to the drive unit without using the SID. The ECG sensors may be implanted, external or both implanted and external. For example, the ECG sensors may be implanted bipolar electrodes positioned at and/or proximate the heart 102 or other appropriate tissue to determine, for example, when the left ventricle is contracting or relaxing. Counterpulsation therapy may be achieved by rapidly inflating the balloon 114 in the aorta 108-109 immediately after aortic valve closure (dicrotic notch) and rapidly deflating the balloon 114 just before the onset of systole using a drive unit. The dicrotic notch may be sensed by the pressure sensor, and the onset of systole may be predicted or sensed using the ECG signals.

The rapid inflation of the balloon 114 may increase the diastolic aortic pressure, improving end-organ perfusion and coronary perfusion. The rapid deflation of the balloon 114 reduces the ejection pressure of the native ventricle, reducing afterload and left ventricular external work. This embodiment of the PiVAS provides counterpulsation therapy in patients that is more effective than a 40-ml IABP device due to a larger displacement volume. The PiVAS has enhanced durability, has a reduced or eliminated risk of being thrombogenic and/or obstructive, and is overall a low-cost, and less invasive device implant/explant procedure without the need to enter the chest. The PiVAS also has low serious adverse event (AE) burden, and enables non-obligatory support to a less-sick heart failure population (the device may be ‘on or off’ as needed).

Balloon and Driveline Embodiments

With reference to FIGS. 5-11 (and FIG. 1 for reference to the aortic arch vasculature 100), various features of a new balloon 502, a new elongated delivery dilator connection element 810, and a new driveline marker element 1102 are disclosed. It is expressly contemplated that aspects and features of the above-described balloon 114, driveline 116, and of the above-described elongated delivery dilator 210, SID and drive unit may be used in or with the features described in FIGS. 5-11, including balloon 502, elongated delivery dilator connection element 810 and driveline marker element 1102. For example, balloon 502 may be composed of a biocompatible, non-thormbogenic elastomeric material (e.g., Biospan®-S) or other suitable materials and may have features described in U.S. Pat. No. 8,066,628.

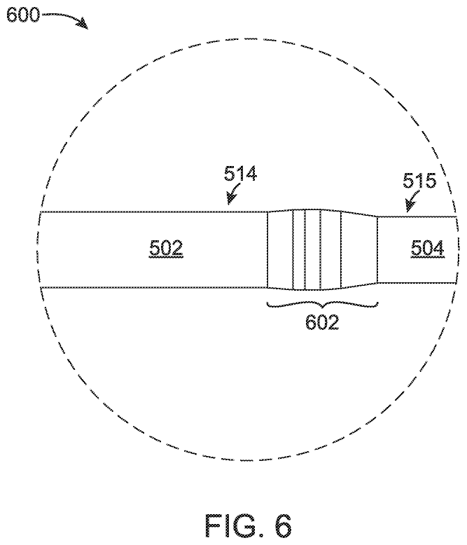

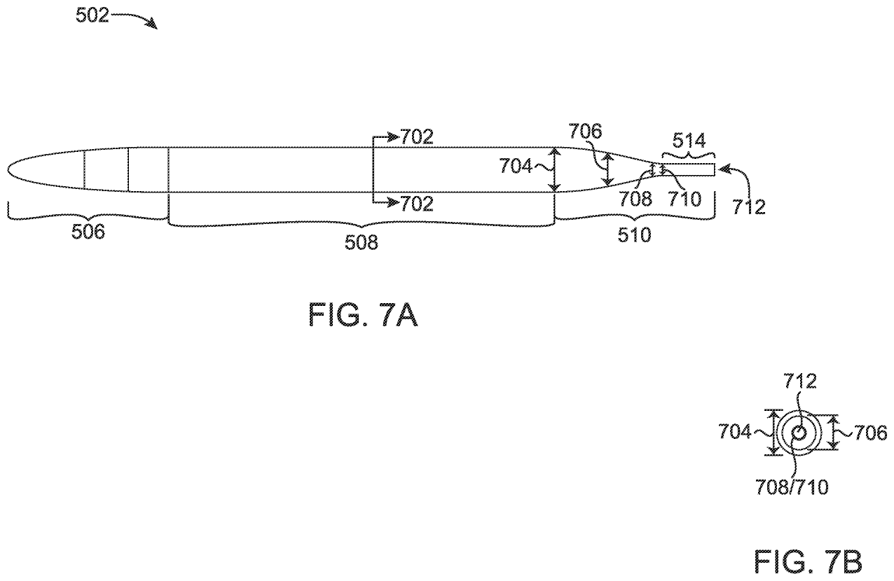

Turning to FIGS. 5-6 where a planar depiction of a balloon pump assembly 500 is illustrated (FIG. 5) and a close up of region 600 (FIG. 6), each in accordance with an embodiment of the present disclosure, the balloon pump assembly 500 may have a balloon 502 and driveline 504. Balloon 502 is an elongated inflatable member with a chamber having a distal region 506, a central region 508, and a proximal region 510. The interior volume of balloon 502 may be between 20 cc and 60 cc (inclusive), and may be between 40 cc and 60 cc (inclusive) for adult populations. The proximal region 510 may be substantially cylindro-conically shaped, tapering toward a proximal end 514 of the balloon 502. A cylindro-conic shape is a conic tank shape. And a shape is substantially cylindro-conically shaped so long as the base is oval, circular, or a shape that deviates only nominally from an oval or circular, and the height of the shape is generally conical. The height can exhibit nominal deviations from a conic shape. For example, so long as proximal region 510 generally resembles a conic tank, then it is substantially cylindro-conically shaped. The proximal region 510 may have an opening at the proximal end 514 (FIG. 7, element 712). In one embodiment, the substantially cylindro-conically shaped proximal region 510 includes a tubular shaped proximal end 514 that defines in part the opening 712.

The central region 508 of balloon 502 may be substantially cylindrically shaped with a substantially uniform exterior diameter 513 dimensioned to be less than the width of a patient's thoracic aorta 108. A shape is substantially cylindrical so long as the base is oval, circular, or a shape that deviates only nominally from an oval or circle, and the height of the shape is generally tubular. The height can exhibit nominal deviations from a tubular shape. For example, so long as central region 508 generally resembles a cylinder or tube, then it is substantially cylindrically shaped. A diameter (here an exterior diameter 513) is “substantially uniform” if the diameter across the applicable region (e.g., the central region 508 of balloon 502) does not deviate more than approximately 0.001 inch (approximately 0.025 mm) across the applicable region (here, balloon 502). In one embodiment, the exterior diameter 513 is approximately 12 mm to 20 mm, inclusive. In one embodiment, the exterior diameter is approximately 17.5 mm.

Distal region 506 of balloon 502 may be substantially cylinder-conically shaped, tapering toward the distal end 512 of balloon 502. The distal end 512 of balloon 502 may be rounded, bullet-shaped, or nipple-shaped.

In one embodiment, the combined length of the proximal region 510 and the central region 508 is so dimensioned such that the combined length is substantially equal to the length of the descending aorta 108-109 from a point proximate the axillary/subclavian arteries 106 to the approximately the two branches of the renal arteries 110. In adults, this length can be between 18-25 cm and in pediatric patients this length can be 10 cm or less. The combined length of the proximal region 510 and the central region 508 is substantially equal to the length of the descending aorta 108-109 from a point proximate the axillary/subclavian arteries 106 to the approximately the two branches of the renal arteries 110 if the combined length of the proximal region 510 and central region 508 is within 95%, 90%, 85%, 80%, 75%, 70%, 65%, 60%, 55%, or 50% of the length of the descending aorta 108-109 from a point proximate the axillary/subclavian arteries 106 to the approximately the two branches of the renal arteries 110. In another embodiment the combined length of the proximal region 510 and the central region 508 is so dimensioned such that the combined length is substantially equal to (i.e., within 95%, 90%, 85%, 80%, 75%, 70%, 65%, 60%, 55%, or 50% of) the length of that portion of the descending aorta 108-109 below or caudal to the axillary/subclavian arteries 106 that has a diameter that is greater than 80%, 85%, 90%, or 95% of the maximum diameter of the thoracic aorta 108. For example, the combined length of the proximal region 510 and the central region 508 may be between approximately 100 mm and 220 mm, inclusive. In one embodiment, the combined length of the proximal region 510 and the central region 508 may be between approximately 160 mm to 220 mm or between approximately 180 mm to 220 mm.

In one embodiment, the tapering of the distal region 506 is substantially consistent with the tapering of the descending aorta 108-109 that begins proximate to or caudal to the two branches of the renal artery 110. Substantially consistent with means that the tapering angle of the distal region 506 is within 10% of the tapering angle of the descending aorta 108-109 proximate to or caudal to the two branches of the renal artery 110). The length of the distal region 506 of the balloon 502 may be greater than approximately 15% of the combined length of the proximal region 510 and the central region 508. In one embodiment, the length of the distal region 506 of balloon 502 is greater than approximately 15% but less than approximately 40% of the combined length of the proximal region 510 and the central region 508. The degree of the tapering may match or exceed the narrowing of the descending aorta 108-109 and may be so dimensioned to not occlude the renal arteries 110 when the balloon 502 is disposed in the descending aorta 108-109 for operation. For the avoidance of doubt, when placed in the descending aorta 108-109, balloon 502 may be disposed such that it spans a greater vertical distance than balloon 114 in FIG. 1. In particular, the distal region 506 may extend past (i.e., caudal to) the two branches of the renal artery 110. In one embodiment, distal region 506 may extend past (i.e., caudal to) the two branches of the renal artery 110 when the proximal region 510 of balloon 502 is disposed in a corresponding position as the proximal region of balloon 114 is depicted in FIG. 1, i.e., when the proximal region 510 of balloon 502 is disposed proximate the aortic arch 104.

In one embodiment, the length of the proximal region 510 is approximately 63 mm, where the portion of the proximal region 510 that is actively tapering is approximately 43 mm and the tubular shaped proximal end 514 is approximately 20 mm. In one embodiment, the central region 508 is approximately 160 mm. And in one embodiment, the distal region 506 is approximately 55 mm. The distal end 512 portion of the distal region 502, which may be rounded, bullet-shaped, or nipple-shaped, may be approximately 30 mm. In one embodiment the entirety of the balloon 502 is approximately 277 mm±2 mm.

In one embodiment, the length of the proximal region 510 is approximately 15-30% of the length of balloon 502. In an embodiment, the length of the central region 508 is approximately 55-65% of the length of balloon 502. In an embodiment, the length of the distal region 512 is approximately 15-30% of the length of the balloon 502.

Balloon 502 may have a wall, the inner surface of such defines an elongated inflatable chamber. A working fluid (e.g., air, gas, etc.) may be delivered to the elongated inflatable chamber to inflate balloon 502 and may be evacuated from the chamber to deflate the balloon 502. The delivery and evacuation of the working fluid may be facilitated by driveline 504. The outer surface of such wall may define the exterior of balloon 502. In one embodiment, the wall of balloon 502 has a substantially uniform thickness. A thickness (here of the wall of balloon 502) is “substantially uniform” if the thickness does not deviate more than approximately 0.001 inch (approximately 0.025 mm) across the applicable region (here, balloon 502). The thickness of the wall of balloon 502 may be between 0.003 inches (0.076 mm) and 0.012 inches (0.305 mm) or 0.014 inches (0.356 mm) thick. In one embodiment, the thickness of the wall of balloon 502 may be between 0.005 inches (0.127 mm) and 0.007 inches (0.178 mm) thick. In other embodiments, the wall of the balloon 502 has a non-uniform thickness. For example, the wall of the balloon 502 at one or more of the distal region 506 and the proximal region 510 (or portions thereof) has a thickness that exceeds that of the central region 508. In yet other embodiments, the wall of the balloon 502 at only the distal end 512 or the proximal end 514 has a thickness that exceeds that of wall of the balloon 502 at the central region 508. In such embodiments, the distal region 506 and proximal region 510 (or portions thereof) or their respective ends 512, 514 may have a wall thicknesses that exceeds the wall thickness of the central region 508. For example, wall thickness at distal region 506 (or a portion thereof) or distal end 512 may be between 0.003 inches (0.076 mm) and 0.016 inches (0.406 mm) thick. In one embodiment, wall thickness at distal region 506 (or a portion thereof) or distal end 512 may be between 0.006 inches (0.152 mm) and 0.009 inches (0.229 mm) thick. Similarly, wall thickness at proximal region 510 (or a portion thereof) or proximal end 514 may be 0.003 inches (0.076 mm) and 0.015 inches (0.381 mm) thick. In one embodiment, wall thickness at proximal region 510 (or a portion thereof) or proximal end 514 may be between 0.006 inches (0.152 mm) and 0.008 inches (0.203 mm) thick.

In embodiments where the wall thickness at distal region 506 (or portion thereof) or distal end 512 exceeds the wall thickness at some portion of the rest of balloon 502, the wall thickness of the distal region 506 (or portion thereof), or distal end 512 may be substantially uniform. In one embodiment, the thicker portion is proximate the central region 508. In another embodiment, a thicker portion is proximate the distal end 512. The wall thickness of central region 508 and/or proximal region 510 may also be substantially uniform.

In embodiments, where the wall thickness at proximal region 510 (or portion thereof) or proximal end 514 exceeds the wall thickness at some portion of the rest of balloon 502, the wall thickness of the proximal region 510 (or portion thereof), or proximal end 514 may be substantially uniform. In one embodiment, the thicker portion proximate the central region 508. In another embodiment, the thicker portion is proximate the proximal end 515. The wall thickness of central region 508 and/or distal region 506 may also be substantially uniform.

In embodiments where the wall thickness at distal region 506 (or portion thereof) or distal end 512 exceeds the wall thickness at some portion of the rest of balloon 502 (e.g., at the central region 508) and where the wall thickness at proximal region 510 (or portion thereof) or proximal end 514 exceeds the wall thickness at some portion of the rest of balloon 502 (e.g., at the central region 508), the wall thickness of the distal region 506 (or portion thereof), or distal end 512 may be substantially uniform, the wall thickness of the proximal region 510 (or portion thereof), or proximal end 514 may be substantially uniform, and the wall thickness of central region 508 and/or proximal region 510 may be substantially uniform.

The increased wall thickness in the proximal region 510 (or portion thereof) or proximal end 514 may minimize strain and kinking at such region (or portion thereof) or end, which might otherwise happen more readily due to the comparatively more stiff driveline 504 relative to the balloon 502. Accordingly, it may be desirable to increase the wall thickness of the proximal region 510 (or portion thereof) or proximal end 514 to minimize the strain placed on this region (or portion thereof) or end of balloon 502 and to prevent such region (or portion thereof) or end of balloon 502 from kinking. Strain and kinking on any portion of balloon 502, including at proximal region 502 (or a portion thereof) or at proximal end 514 may, if not minimized, reduced, or eradicated, may cause balloon failure.

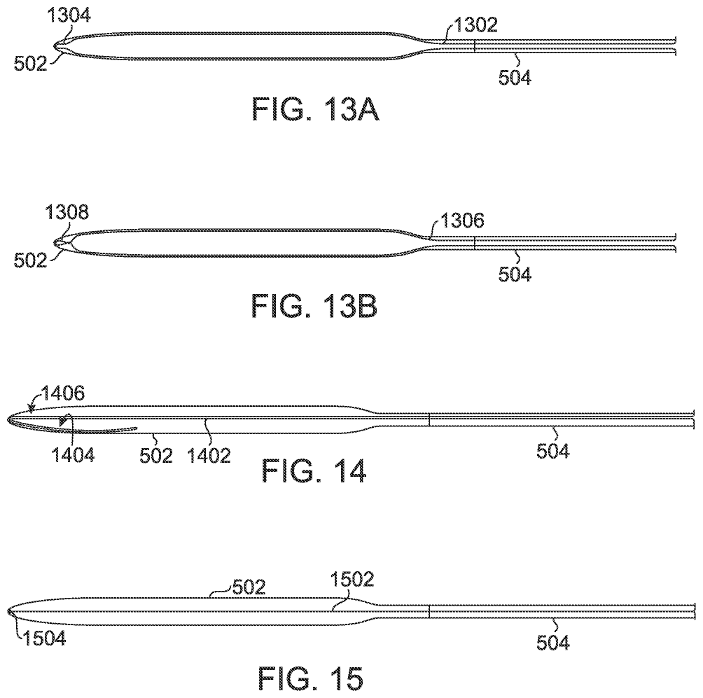

The increased wall thickness in the distal region 506 or distal end 512 may minimize strain on the balloon 502 in an embodiment where the balloon 512 is sized and shaped to receive a support structure configured to oppose buoyancy forces acting on balloon 502 during operation in the descending aorta 108-109. Such buoyancy forces might otherwise cause balloon 502 to roll or fold upon itself (or otherwise cause crevices to develop in balloon 502) along one or more of a longitudinal axis of the balloon 502 or a latitudinal axis of the balloon 502 as is described in the U.S. Patent Application filed on the same date as the present disclosure, owned by Applicant/Assignee NuPulseCV, Inc., entitled “A Blood Pump Support Apparatus and Method for a Blood Pump Assembly,” identifying Robert Christopher Hall, Joshua Ryan Woolley, Guruprasad Anapathur Giridharan, and Duane Sidney Pinto as inventors, and having attorney-docket number 8020US00/236533-30035, the contents of which are incorporated by reference in its entirety. Such a support structure may be implanted into the blood pump assembly 500, and in particular the balloon 502. Exemplary support structures may take on various shapes including a wire loop, a wire with a flexible tip portion that when disposed in balloon 502 is curled back on some portion of the wire support structure, and a wire with a blunted distal end, as is generally depicted in FIGS. 13-15. FIGS. 13A and 13B depict wire loop support structures 1302 and 1306, respectively, disposed within balloon 502. The distal end of the support structure 1302 includes a nipple 1304 formed as a pinch point in the wire loop support structure whereas the distal end of the wire loop support structure 1306 includes a nipple 1308 formed by the wire crossing over itself (e.g., the wire is twisted on itself). FIG. 14 depicts wire support structure 1402 with a flexible tip portion 1404 and a non-flexible body portion 1406 disposed within balloon 502. The flexible tip portion 1404 may comprise a tail. FIG. 15 depicts wire support structure 1502 with a blunted distal end 1504 disposed within balloon 502. In such embodiments, the support structure (e.g., wire support structures 1302, 1306, 1402 and 1502) may operationally apply a force and a strain on the wall of balloon 502, in particular at the distal region 506 (or portion thereof) or distal end 512 or balloon 502, and without the increased thickness the balloon 502 may fail. In the embodiment of wire loop support structures 1302, 1306, forces and strain may be caused in part by the nipples 1304, 1308 and/or the distal region of the wire loop itself. Similarly, in the embodiment of a wire support structure 1402, the forces and strain may be caused in part by the flexible tip portion 1404 (e.g., where the flexible tip portion 1404 meets the non-flexible body portion 1406 and/or along the curled back or tail portion of the flexible tip portion 1404). And in the embodiment of a wire support structure 1502, the forces and strain may be caused in part by the blunted distal tip 1504.

Wall thickness may be related to the width of an inner lumen of the delivery dilator and/or introducer sheath 204, 206. In an embodiment where the distal region 506 (or portion thereof) or the distal end 512 tapers, the tapering may permit manufacture of a balloon 502 with a relatively thicker wall in such tapered region as compared to an embodiment with no such tapering or less tapering. Similarly, in an embodiment where the proximal region 510 (or portion thereof) or the proximal end 514 tapers, the tapering may permit manufacture of a balloon 502 with a relatively thicker wall in such tapered region as compared to an embodiment with no such tapering or less tapering.

Blood pump assembly 500 may also include driveline 504 having a distal region 515 and a proximal end 516, the distal end 515 of driveline 504 coupleable to the proximal end 514 of balloon 502 at a joint 602. The joint 602 may be formed at an engagement region at the proximal end 514 of balloon 502 that is sized and shaped to fit over a distal end 515 of driveline 504. An attachment feature such as a compression ring or other suitable element providing an airtight connection/pneumatic seal between the engagement region and driveline 504 may be used to couple the balloon 502 to the driveline 504. In one embodiment, a BioSpan®-S coating may be applied to joint 602 to produce a smooth joint that promotes laminar fluid flow.

Although not depicted, in one embodiment balloon 502, when properly disposed for operation, will extend beyond the thoracic aorta 108 and will be disposed in descending aorta 108-109.

FIGS. 7A and 7B illustrate a planar illustration and transverse cross section illustration of balloon 502 in accordance with one embodiment, where FIG. 7A is the planar illustration and FIG. 7B is the transverse cross section taken at section line 702 facing the proximal region 510 of balloon 502 and illustrating the substantially cylindro-conically shaped proximal region 510, tapering toward a proximal end 514 of the balloon 502 at opening 712. The central region 508 of balloon 508 may have a substantially uniform inner diameter 704. The inner diameter of the proximal region 510 may taper to progressively smaller inner diameters 706, 708, 710, consistent with the tapering of the of the descending aorta 108-109 that begins proximate to or caudal to the two branches of the renal artery 110. In one embodiment, inner diameter 710 is the inner diameter of the optional tubular shaped proximal end 514 and the inner diameter 710 of opening 712. In one embodiment, the inner diameter 710 is approximately 4.35 mm.

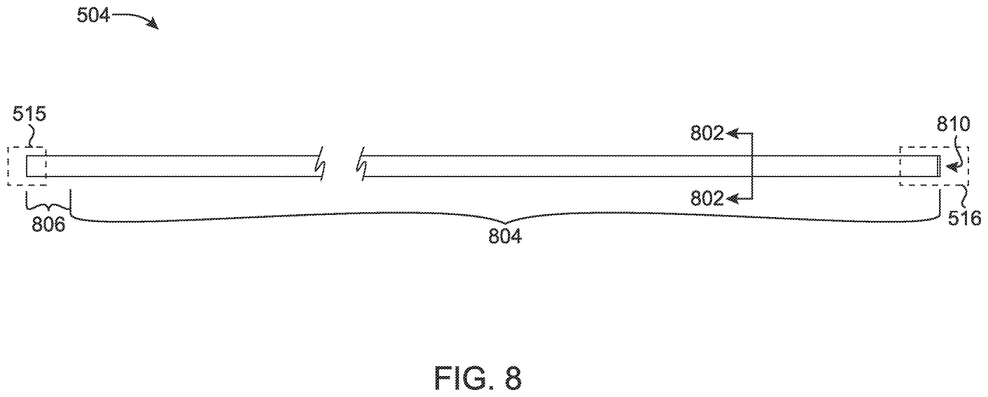

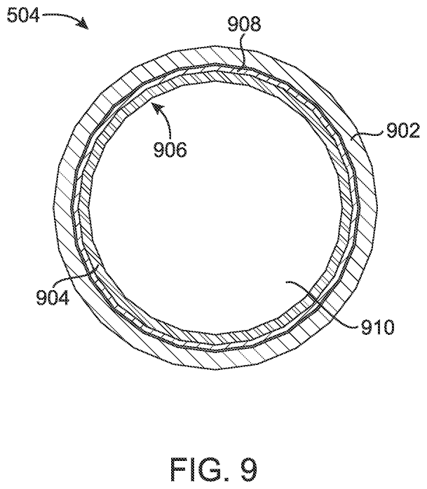

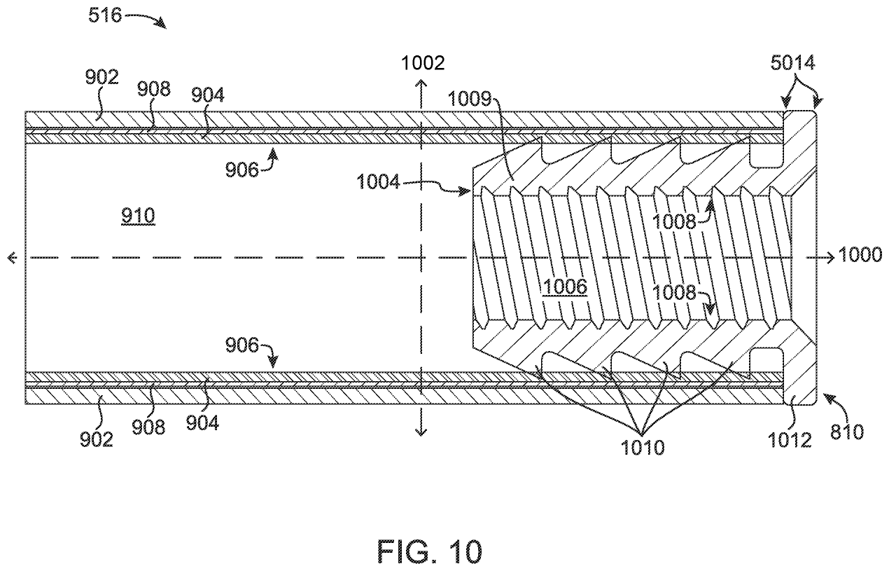

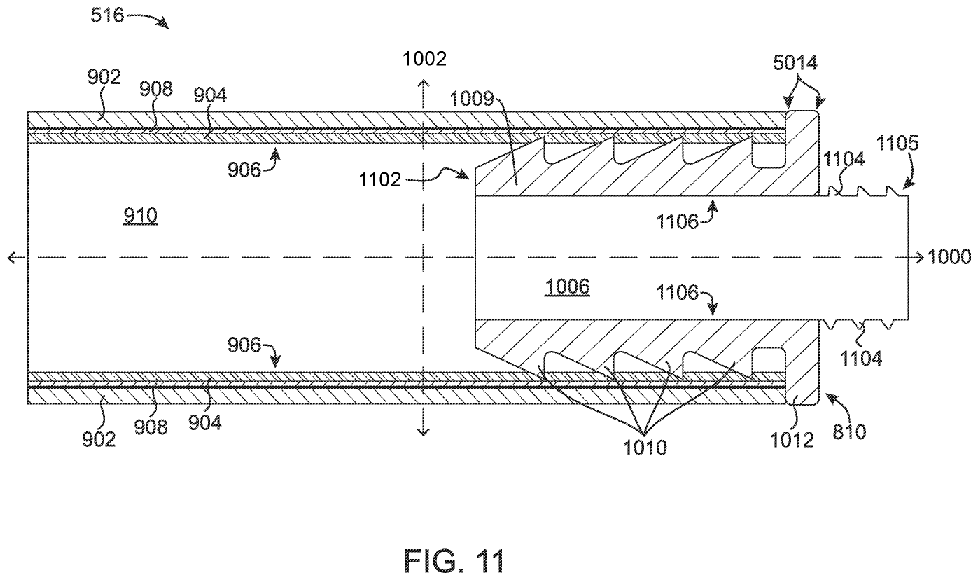

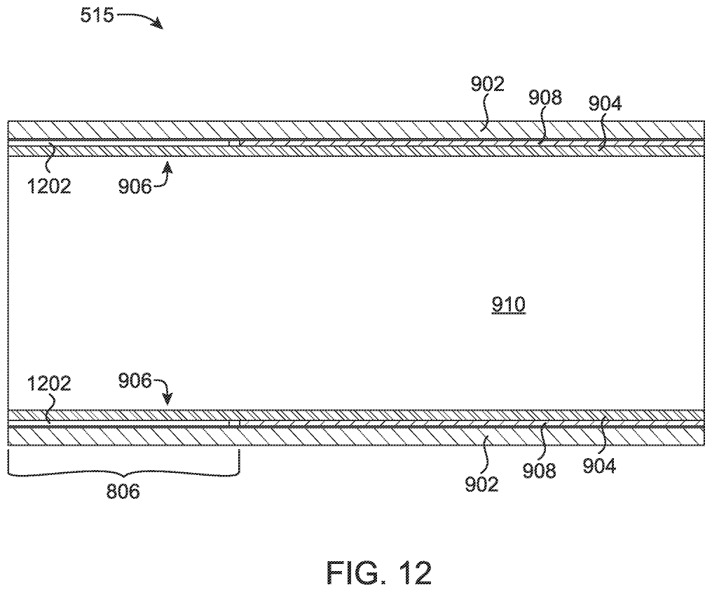

FIGS. 8-12 depicts features of driveline 504, including exemplary new elongated delivery dilator connection element 810 and new driveline marker element 1202. FIG. 8 illustrates a planar depiction of driveline 504. FIG. 9 illustrates a transverse cross-section of driveline 504 taken at section 802 and facing distal end 515 of driveline 504. FIGS. 10-11 illustrate a longitudinal cross section of driveline 504 at proximal region 516, illustrating two exemplary elongated delivery dilator connection elements 810. And FIG. 12 illustrates a longitudinal cross section of driveline 504 at distal end 515 illustrating driveline marker element 1202. With reference to FIGS. 8-12, driveline 504 may comprise an outer membrane 902, inner membrane 904 and kink-resistant element 908 that may be identical to the similarly-named components 408, 406, and 410 of FIGS. 4B-C. Inner membrane 904 may have an inner wall 906 that defines lumen 910. Lumen 910 may be identical to lumen 404 of FIG. 4B. Kink-resistant element 908 may be disposed in between outer membrane 902 and inner membrane along a first length 804 of driveline 504.

Elongated delivery dilator connection element 810 may be disposed at the proximal end 516 of driveline 502 and may be configured to be sized and shaped to interface with a corresponding driveline connection element associated with the distal end of an elongated delivery dilator (e.g., driveline connection element 310B). For example, the elongated delivery dilator connection element 810 may include (as depicted in FIG. 10) a threaded female connection element and a corresponding driveline connection element (e.g., element 310B) may include a threaded male connection element, such threaded female connection element is sized and shaped to receive such threaded male connection element. In another example, the elongated delivery dilator connection element 810 may include a threaded male connection element (as depicted in FIG. 11) and a corresponding driveline connection element may include a threaded female connection element, such threaded male connection element being sized and shaped to receive such threaded female connection element. Using the delivery dilator 210 as an exemplary delivery dilator, distal end 212 of delivery dilator 210 may be connected to proximal end 516 of driveline 504 by screwing the threaded male connection element into the threaded female connection element.

The elongated delivery dilator connection element 810 may not be threaded. Instead, connection element 810 may include other connection elements such as snap fit connections.

The elongated delivery dilator connection element 810 may include a channel 1006 that is in fluid communication with lumen 910. Such fluid connection may be utilized to de-air the balloon 502 during implantation and/or extraction. In some embodiments, channel 1006 may also be used during operation of balloon 502 (e.g., if driveline 504 is not re-sized after implantation).

The elongated delivery dilator connection element 810 may be secured to the proximal end 516 of driveline 504 using means such that the separation force required to separate the elongated delivery dilator connection element 810 from proximal end 516 of driveline 504 exceeds at least approximately 8 pounds-force. In other embodiments, the separation force exceeds 12 to 30 pounds-force. In one embodiment, the elongated delivery dilator connection element 810 may be a hose barb fitting 1004, 1102 having a threaded connection element such as hose barb fitting 1004 having a threaded female connection element 1108 or hose barb fitting 1102 having a threaded male connection element 1104.

The hose barb fitting 1004, 1102 may be disposed at least partially within the driveline 504 at proximal end 516. With reference to a longitudinal axis 1000 and a transverse axis 1002, hose barb fitting 1004, 1102 may include an interior surface 1008 or 1106 defining channel 1006 disposed along, along an angle with respect to, or substantially along (e.g., within an angle that is within 45 degrees of) the longitudinal axis 1000, at least one barb 1010 extending circumferentially outward from the interior surface 1008, 1106, and defining an exterior surface 1009. The at least one barb 1010 extending outward along, along an angle with respect to, or substantially along (e.g., within an angle that is within 45 degrees of) the transverse axis 1002, and the at least one barb 1010 being sized and shaped to be embedded into an inner wall 906 of driveline 504. In one embodiment, the at least one barb 1010 is sized and shaped to be embedded into the inner membrane 904 of driveline 504. In one embodiment, the at least one barb 1010 is sized and shaped to be embedded into the kink-resistant element 908 and optionally the outer membrane 902.

In addition to defining channel 1006, the interior surface 1008 of the hose barb fitting 1004 may be threaded so as to form the threaded female connection element. Whereas, the interior surface 1106 in hose barb fitting 1102 may not be threaded, but may define channel 1106 and the interior surface of bolt 1105 that extends out from the proximate end 516 of driveline 504. The exterior surface of bolt may be threaded 1104 so as to form the threaded make connection element.

Hose barb fitting 1004, 1102 may include a flange 1012 disposed at the proximal end of the hose barb fitting 1004, 1102 and may extend circumferentially outward substantially along (e.g., within an angle that within 45 degrees of) the transverse axis 1002. Flange 1012 may extend circumferentially outward along, along an angle with respect to, or substantially along (e.g., when an angle that is within 45 degrees of) the transverse axis 1002, and extending substantially to the outer edge of the proximal end 516 of the driveline 504, and be sized and shaped to seal lumen 910 and otherwise be configured to be mated with the exterior surface of a driveline connection element associated with an elongated delivery dilator (e.g., driveline connection element 310B). For example, the exterior of the flange 1012 may be configured to mate flush with the delivery dilator (e.g., the corresponding driveline connection element thereof) to minimize blood intrusion while being pulled through the vasculature and to maintain a vacuum or a substantial vacuum (i.e., conditions that are within 95%, 90%, 85%, 80%, 75%, 70%, 65%, 60%, 55%, or 50% of a vacuum) after balloon 502 is de-aired. In one embodiment, the exterior facing surface of flange 510 is rounded at the outer-most transverse edges 5014 (both distal and proximate to the proximate end 516 of driveline wall) to mitigate against vascular injury when the driveline 504 is being pulled through the patient's vasculature. In one embodiment, the elongated delivery dilator connection element 810 is further glued to the inside of the driveline 504 (e.g., at inner wall 906). In one embodiment, heat shrink elements (not depicted) may be used on the exterior surface of driveline 504 at the proximate end 516 to further secure elongated delivery dilator connection element 810 to driveline 504. Although depicted with kink-resistant element 908 extending to the proximal edge of proximal end 516, in one embodiment, kink-resistant element 908 extents only to the distal edge of the elongated delivery dilator connection element 810.

The inner diameter of driveline 504 may be substantially consistent from distal end 515 to proximal end 516, including in that portion of the proximal end 516 where elongated delivery dilator connection element 810 is disposed and secured. That is, the deviation of the inner diameter across the driveline is less than or equal to 10%. A substantially consistent inner diameter may facilitate consistent flow of the working fluid into and out of the balloon 502 during inflation and deflation.

Although not specifically illustrated as such, driveline connection element 310B may be secured to the distal end 212 of the delivery dilator 210 using means such that the separation force required to separate the driveline connection element from distal end 212 of delivery dilator 210 exceeds at least approximately 8 pounds-force. In other embodiments, the separation force exceeds 12 to 20 pounds-force. In one embodiment, the driveline connection element may be a hose barb fitting having a threaded connection element such as hose barb fitting having a threaded female connection element or hose barb fitting having a threaded male connection element. The hose barb fitting may take the same structure as hose barb fitting 1104, 1102.

As described above kink-resistant element 908 may be disposed in between outer membrane 902 and inner membrane along a first length 804 of driveline 504. The first length 804 may span a majority of the longitudinal distance of driveline 504. In one embodiment, first length 804 extends from the proximal edge of proximal end 516 to a region within or proximate the distal end 515 of driveline 504. In another embodiment, first length 804 extends substantially (i.e., within 95%, 90%, 85%, 80%, 75%, 70%, 65%, 60%, 55% or 50%) from the distal edge of the elongated delivery dilator connection element 810 to a region within or proximate the distal end 515 of driveline 504. In another embodiment, first length 804 may extend less than a majority of the longitudinal distance of driveline 504 or may intermittently cover such distance. First length 804 of driveline may be any length of driveline 504 and may not be contiguous.

A radio opaque material 1202 may be sandwiched between outer membrane 902 and inner membrane 904 and disposed along a second length 806 of driveline 504. The radio opaque material may also be positioned on the outside of the driveline 504 and fixated with a BioSpan®-S coating or applied as part of joint 602 to produce a smooth surface that promotes laminar fluid flow. Radio opaque material 1202 may act as a marker suitable to track the position of driveline 504 (and thus the balloon 502) during radiological intervention (e.g., on an x-ray or fluoroscopy). Second length 806 of driveline 504 may extend from substantially (i.e., within 95%, 90%, 85%, 80%, 75%, 70%, 65%, 60%, 55%, or 50%) the distal end of the distal edge 515 of driveline 504 to a region proximate the distal end of the first length 804. In another embodiment, second length 806 of driveline 504 may extend from substantially (i.e., within 95%, 90%, 85%, 80%, 75%, 70%, 65%, 60%, 55%%, or 50%) the distal end of the distal edge 515 of driveline 504 to the proximate edge of the distal region 515 of driveline 504. In another embodiment, second length 806 of driveline 504 may extend from substantially (i.e., within 95%, 90%, 85%, 80%, 75%, 70%, 65%, 60%, 55%%, or 50%) the distal end of the distal edge 515 of driveline 504 to a point proximate the middle of the distal region 515 of driveline 504. In one embodiment, second length 806 may be any length of driveline 504 and may not be contiguous, extend to the distal edge of distal end 515 of driveline 504 or be limited to the distal end 515 of driveline 504. In one embodiment, second length 806 may be the width of a radio opaque marker 1202. In one embodiment, first length 804 and second length 806 may constitute substantially (i.e., within 95%, 90%, 85%, 80%, 75%, 70%, 65%, 60%, 55%%, or 50%) the length of driveline 504.

Radio opaque material 1202 may be a band of radio opaque material. And in one embodiment marker 1202 may have substantially the same thickness as the kink-resistant element 908 such that the driveline 502 inner diameter and external diameter is substantially uniform throughout the length of driveline 502. In other embodiments, the radio opaque material 1202 may be a coil. Radio opaque material 1202 may be continuous or interrupted.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. The various embodiments described herein may also be combined to provide further embodiments. For example, the above embodiments are not mutually exclusive and the features depicted in any such embodiment may be combined with features of other embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology.

Where the context permits, singular or plural terms may also include the plural or singular term, respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. Further, any ranges identified herein are intended to be inclusive of the numbers that define the range, whether expressly stated or not. The same applies to approximate ranges. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology may encompass other embodiments not expressly shown or described herein.

From the foregoing, it will be appreciated that specific embodiments of the invention have been described herein for purposes of illustration, but that various modifications may be made without deviating from the scope of the invention. Accordingly, the invention is not limited except as by the appended claims.

The foregoing technology solves multiple problems with the prior art. The particular configuration of balloon 502 is designed to match the configuration of the descending aorta 108-109 without obstructing descending the aorta 108-109 itself of any branching vessels while enabling additional volume for greater support for the heart. The reinforced driveline 504 and new connector elements (e.g., elongated delivery dilator connection element 810) provides reinforcements that were lacking in prior art/conventional designs. These reinforcements permit navigation of the tortuous curvature from the axillary artery to the thoracic aorta (or vice-versa). Finally, the integrated radio opaque material 1202 solves various problems associated with conventional radio opaque markers.

We claim:

1. A blood pump assembly comprising:
a balloon defining an elongated inflatable chamber having an interior volume, the balloon having a distal region with a distal end, a central region, and a proximal region with a proximal end, wherein the proximal end of the balloon has an opening; and
wherein:
the interior volume of the inflatable chamber is between approximately 20 cc and 60 cc, inclusive,
the proximal region of the balloon is substantially cylindro-conically shaped, the proximal region of the balloon tapering toward the proximal end of the balloon,
the central region of the balloon is substantially cylindrically shaped with a substantially uniform exterior diameter of approximately 12 mm to 20 mm, inclusive,
the distal region of the balloon is substantially cylindro-conically shaped, the distal region of the balloon tapering toward the distal end of the balloon,
the combined length of the proximal and central regions of the balloon is between approximately 100 mm and 220 mm, inclusive, and
the length of the distal region of the balloon is greater than approximately 15% of the combined length of the proximal and central regions of the balloon.

2. The blood pump assembly of claim 1, wherein the distal end of the balloon is rounded.

3. The blood pump assembly of claim 1, wherein the distal end of the balloon is nipple-shaped.

4. The blood pump assembly of claim 1, wherein the distal end of the balloon is bullet-shaped.

5. The blood pump assembly of claim 1, wherein the length of the distal region of the balloon is less than approximately 40% of the combined length of the proximal and central regions of the balloon.

6. The blood pump assembly of claim 1, further comprising a driveline having a distal end, a proximal end, a central lumen configured to communicate a working fluid for delivery to or from the elongated inflatable chamber, and a connection element, wherein:
the distal end of the driveline is coupleable to the proximal end of the balloon at the opening,
the connection element is disposed at and secured to the proximal end of the driveline, wherein the separation force required to separate the connection element from the proximal end of the driveline exceeds at least approximately 12 to 30 pounds-force,
the connection element is sized and shaped to receive a corresponding connection element associated with a delivery dilator configured to pull the driveline through a patient's vasculature, and
the connection element has a channel configured to permit the fluid to pass there though and into or out of the central lumen.

7. The blood pump of claim 6 wherein the driveline has an outer diameter that is substantially uniform.

8. The blood pump assembly of claim 6 wherein:
the connection element is a hose barb fitting disposed at least partially within the driveline at the proximal end of the driveline, and
the hose barb fitting includes:
a longitudinal axis, a transverse axis, at least one barb on an exterior surface, an interior surface defining the channel, and a flange,
the channel is disposed substantially along the longitudinal axis,
the at least one barb extends circumferentially outward from the interior surface substantially along the transverse axis, the at least one barb being sized to be embedded into an inner wall of the driveline, and
a flange disposed at the proximal end of the hose barb fitting and extending circumferentially outward and substantially along the transverse axis, the flange being located external to the driveline and extending substantially to the outer edge of the proximal end of the driveline, an exterior-facing surface of the flange being configured to be mated with an exterior surface of the corresponding connection element associated with the delivery dilator.

9. The blood pump assembly of claim 8, wherein the exterior-facing surface of the flange is:
rounded at the outer-most edge to mitigate against vascular injury when the driveline is being pulled through the patient's vasculature, and
shaped and sized to mate flush with and at least substantially create a vacuum with the exterior surface of the corresponding connection element associated with the delivery dilator.

10. The blood pump assembly of claim 8, wherein:
the driveline includes an outer membrane, an inner membrane, and a kink-resistant element disposed between the outer membrane and the inner membrane along a first length of the driveline, and
the at least one barb is embedded into the inner membrane of the driveline.

11. The blood pump assembly of claim 10, wherein the kink-resistant element is a helically wound Nitinol coil.

12. The blood pump assembly of claim 10, wherein the outer membrane comprises a biocompatible and anti-thrombogenic material.

13. The blood pump of claim 10, wherein:
radio opaque material is disposed between the outer membrane and the inner membrane along a second length of the driveline, and
the second length of the driveline is proximate to or at the distal end of the driveline.

14. The blood pump of claim 13, wherein the radio opaque material is a ring.

15. The blood pump assembly of claim 13, wherein the radio opaque material has substantially the same thickness as the kink-resistant element.

16. The blood pump assembly of claim 1, further comprising a driveline having a distal end, a proximal end, an outer membrane and an inner membrane, and a central lumen, wherein:
a kink-resistant element is disposed between the outer membrane and the inner membrane along a first length of the driveline, and
radio opaque material is disposed between the outer membrane and the inner membrane along a second length of the driveline.

17. The blood pump assembly of claim 16, wherein the second length of the driveline is proximate to or at the distal end of the driveline.

18. The blood pump assembly of claim 16, wherein the radio opaque material has the substantially the same thickness as the kink-resistant element.

19. The blood pump assembly of claim 16, wherein the radio opaque material is a ring.

20. The blood pump assembly of claim 1, wherein the substantially cylindro-conically shaped proximal region of the balloon includes a tubular-shaped proximal end.

21. The blood pump assembly of claim 1, wherein the balloon has a wall, the inner surface of said wall defining the elongated inflatable chamber, the wall having a non-uniform thickness.

22. The blood pump assembly of claim 21, wherein:
the driveline has a stiffness, and
the wall thickness at a portion of the proximal region of the balloon exceeds the wall thickness at the central region to minimize at least one of strain on or kinking in said portion of the proximal region of the balloon during operation of the blood pump assembly caused by the driveline stiffness.

23. The blood pump assembly of claim 21, wherein:
the balloon is sized and shaped to receive a blood pump support structure configured to oppose buoyancy forces acting on the balloon when the blood pump assembly is implanted in the descending aorta, and
the wall thickness at a portion of the distal region of the balloon exceeds the wall thickness at the central region of the balloon such that during operation of the blood pump assembly, a strain on said portion of the distal region of the balloon caused by the blood pump support structure is minimized.

24. A blood pump assembly comprising:
a balloon defining an elongated inflatable chamber having an interior volume, the balloon having a distal region with a distal end, a central region, and a proximal region with a proximal end, wherein the proximal end of the balloon has an opening; and
wherein:
the interior volume of the elongated inflatable chamber is between approximately 20 cc and 60 cc, inclusive,
the proximal region of the balloon is substantially cylindro-conically shaped, the proximal region of the balloon tapering toward the proximal end of the balloon, the proximal region being approximately 15-30% of the length of the balloon,
the central region of the balloon is substantially cylindrically shaped with a substantially uniform exterior diameter, the central region being approximately 55-65% of the length of the balloon, and
the distal region of the balloon is substantially cylindro-conically shaped, the distal region of the balloon tapering toward the distal end of the balloon, the distal region being approximately 15-30% of the length of the balloon.

25. The blood pump assembly of claim 24, wherein:
the combined length of the proximal and central regions of the balloon is between approximately 100 mm and 220 mm, inclusive, and

26. The blood pump assembly of claim 24, wherein the distal end of the balloon is one of: rounded, nipple-shaped, and bullet-shaped.

27. The blood pump assembly of claim 24, further comprising a driveline having a distal end, a proximal end, a central lumen configured to communicate a working fluid for delivery to or from the elongated inflatable chamber, and a connection element, wherein:
the distal end of the driveline is coupleable to the proximal end of the balloon at the opening,
the connection element is disposed at and secured to the proximal end of the driveline, wherein the separation force required to separate the connection element from the proximal end of the driveline exceeds at least approximately 12 to 30 pounds-force,
the connection element is sized and shaped to receive a corresponding connection element associated with a delivery dilator configured to pull the driveline through a patient's vasculature, and
the connection element has a channel configured to permit the fluid to pass there though and into or out of the central lumen.

28. The blood pump assembly of claim 27, wherein:
the connection element is a hose barb fitting disposed at least partially within the driveline at the proximal end of the driveline, and
the hose barb fitting includes:
a longitudinal axis, a transverse axis, at least one barb on an exterior surface, an interior surface defining the channel, and a flange,
the channel is disposed substantially along the longitudinal axis,
the at least one barb extends circumferentially outward from the interior surface substantially along the transverse axis, the at least one barb being sized to be embedded into an inner wall of the driveline, and
a flange disposed at the proximal end of the hose barb fitting and extending circumferentially outward and substantially along the transverse axis, the flange being located external to the driveline and extending substantially to the outer edge of the proximal end of the driveline, an exterior-facing surface of the flange being configured to be mated with an exterior surface of the corresponding connection element associated with the delivery dilator.

29. The blood pump assembly of claim 28, wherein:
the driveline includes an outer membrane, an inner membrane, and a kink-resistant element disposed between the outer membrane and the inner membrane along a first length of the driveline, and
the at least one barb is embedded into the inner membrane of the driveline.

30. The blood pump of claim 29, wherein:
radio opaque material is disposed between the outer membrane and the inner membrane along a second length of the driveline, and
the second length of the driveline is proximate to or at the distal end of the driveline.

31. The blood pump assembly of claim 30, wherein the radio opaque material has substantially the same thickness as the kink-resistant element.

32. The blood pump assembly of claim 24, further comprising a driveline having a distal end, a proximal end, an outer membrane and an inner membrane, and a central lumen, wherein:
a kink-resistant element is disposed between the outer membrane and the inner membrane along a first length of the driveline, and
radio opaque material is disposed between the outer membrane and the inner membrane along a second length of the driveline.

33. The blood pump assembly of claim 32, wherein:
the second length of the driveline is proximate to or at the distal end of the driveline,
the radio opaque material has the same thickness as the kink-resistant element, and
the radio opaque material is a ring.

34. The blood pump assembly of claim 24, wherein the balloon has a wall, the inner surface of said wall defining the elongated inflatable chamber, the wall having a non-uniform thickness.

35. The blood pump assembly of claim 34, wherein:
the driveline has a stiffness, and
the wall thickness at a portion of the proximal region of the balloon exceeds the wall thickness at the central region to minimize at least one of strain on or kinking in said portion of the proximal region of the balloon during operation of the blood pump assembly caused by the driveline stiffness.

36. The blood pump assembly of claim 34, wherein:
the balloon is sized and shaped to receive a blood pump support structure, configured to oppose buoyancy forces acting on the balloon when the blood pump assembly is implanted in the descending aorta, and
the wall thickness at a portion of the distal region of the balloon exceeds the wall thickness at the central region of the balloon such that during operation of the blood pump assembly, a strain on said portion of the distal region of the balloon caused by the blood pump support structure is minimized.
